(19) United States
(12) Patent Application Publication
(10) Pub. No.: US 2005/0131344 A1
(43) Pub. Date: Jun. 16, 2005

(54) Low-profile valve contained within a catheter lumen

(75) Inventor: Raymond E. Godaire, Auburn, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC.

(21) Appl. No.: 10/736,149

(22) Filed: Dec. 15, 2003

(51) Int. Cl. A61M 29/00

(52) U.S. Cl. 604/99.04

(57) ABSTRACT

A low-profile catheter valve includes a catheter, an elastomeric plug having a channel, and a hollow stem. The catheter has at least one stop extending into a central lumen of the catheter adjacent to the catheter's proximal end. The elastomeric plug is received within the central lumen of the catheter and seated against the stop. The hollow stem is slidably received within the central lumen of the catheter. An axial force applied to the elastomeric plug via the stem compresses the plug and closes the channel. The stem is maintained in a position within the catheter by an interference fit between the stem and the catheter.

### TECHNICAL FIELD

This invention relates generally to biomedical devices that are used for treating vascular conditions. More specifically, the invention relates to a low-profile catheter valve that is contained within the central lumen of a catheter used as a guidewire.

### BACKGROUND OF THE INVENTION

Guidewires are conventionally used to guide medical instruments to a desired treatment location within a patient's vasculature. In a typical procedure, the clinician forms an access point for the guidewire by creating an opening in a peripheral blood vessel, such as the femoral artery. The highly flexible guidewire is then introduced through the opening and is advanced by the clinician through the patient's blood vessels until the guidewire extends across a vessel segment to be treated. A treatment catheter, such as a balloon catheter for a percutaneous transluminal coronary angioplasty (PTCA), may then be inserted over the guidewire and similarly advanced through vasculature until it reaches the treatment site.

In certain treatment procedures, it is desirable to serially advance and withdraw a number of different treatment catheters over a single guidewire that has been placed in a particular location. Typically, a first treatment catheter is advanced over the guidewire, withdrawn, and then fully removed from the portion of the guidewire that extends out of the patient's vessel. The guidewire is then available to act as a guide for a different treatment catheter.

It is sometimes advantageous to equip the distal end of a guidewire with at least one inflatable balloon, either to provide temporary occlusion of a vessel or to anchor the guidewire within a vessel. Anchoring the guidewire helps to prevent the guidewire from being displaced from its position while treatment catheters are advanced or withdrawn over the placed guidewire. An occlusion guidewire can be used as “distal protection” to prevent debris generated during vessel treatment from moving with the flowing blood to embolize distally.

A permanent inflation manifold, of the type used with conventional catheters having an inflatable balloon, would prevent treatment catheters from being exchanged one for another over an occlusion guidewire. Therefore, a removable inflation manifold and a valve to maintain the balloon in the inflated state are desirable for an occlusion guidewire. U.S. Pat. No. 5,167,239 to Cohen et al. discloses one such device. However, the valve apparatus used by the Cohen device is relatively bulky, having an outer diameter in its preferred embodiment of 0.0355 inches. As can be readily appreciated, the diameter of the valve on a guidewire dictates the inner diameter and, consequently, the outer diameter of a treatment catheter introduced over the valve. Therefore, it would be desirable to provide a low-profile catheter valve that overcomes the aforementioned and other disadvantages.

### SUMMARY OF THE INVENTION

One aspect of the present invention is a low-profile catheter valve, comprising a catheter, an elastomeric plug, and a hollow stem. The catheter has at least one stop extending into the central lumen of the catheter adjacent to the catheter's proximal end. At least a portion of the elastomeric plug is received within the central lumen of the catheter and seated against the stop. The hollow stem is slidably received within the central lumen of the catheter.

The elastomeric plug has a channel in fluid communication with the central lumen of the catheter. An axial force applied to the elastomeric plug via the stem compresses the plug and closes the channel. The stem is maintained in a position within the catheter by interference between the stem and the catheter.

Another aspect of the present invention is a system for treating a vascular condition, comprising a catheter, an elastomeric plug, a hollow stem, and an inflatable balloon. The catheter has at least one stop extending into the central lumen of the catheter adjacent to the catheter's proximal end. At least a portion of the elastomeric plug is received within the central lumen of the catheter and seated against the stop. The hollow stem is slidably received within the central lumen of the catheter. The balloon is operably attached to a distal portion of the catheter.

The elastomeric plug has a channel in fluid communication with the central lumen of the catheter that allows inflation or deflation of the balloon through the central lumen. An axial force applied to the elastomeric plug via the stem compresses the plug and closes the channel to maintain inflation of the balloon. The stem is maintained in a position within the catheter by interference between the stem and the catheter.

The aforementioned and other features and advantages of the invention will become further apparent from the following detailed description of the presently preferred embodiments, read in conjunction with the accompanying drawings, which are not to scale. The detailed description and drawings are merely illustrative of the invention rather than limiting, the scope of the invention being defined by the appended claims and equivalents thereof.

### BRIEF DESCRIPTION OF THE DRAWINGS

FIGS. 1A and 1B show longitudinal cross-sectional views of one embodiment of a low-profile catheter valve, in accordance with the present invention;

FIGS. 2A and 2B show longitudinal cross-sectional views of another embodiment of a low-profile catheter valve, in accordance with the present invention;

FIG. 3 is a longitudinal cross-sectional view of an adaptor used to manipulate the low-profile catheter valve of FIG. 1, showing the adaptor in a first position; and

FIG. 4 is an illustration of one embodiment of a system for treating a vascular condition, in accordance with the present invention;

### DETAILED DESCRIPTION OF THE PRESENTLY PREFERRED EMBODIMENTS

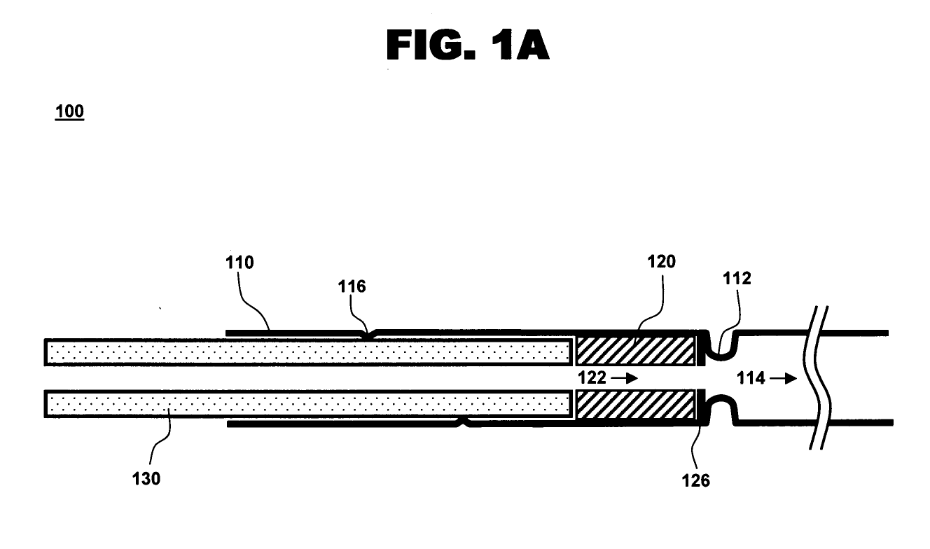

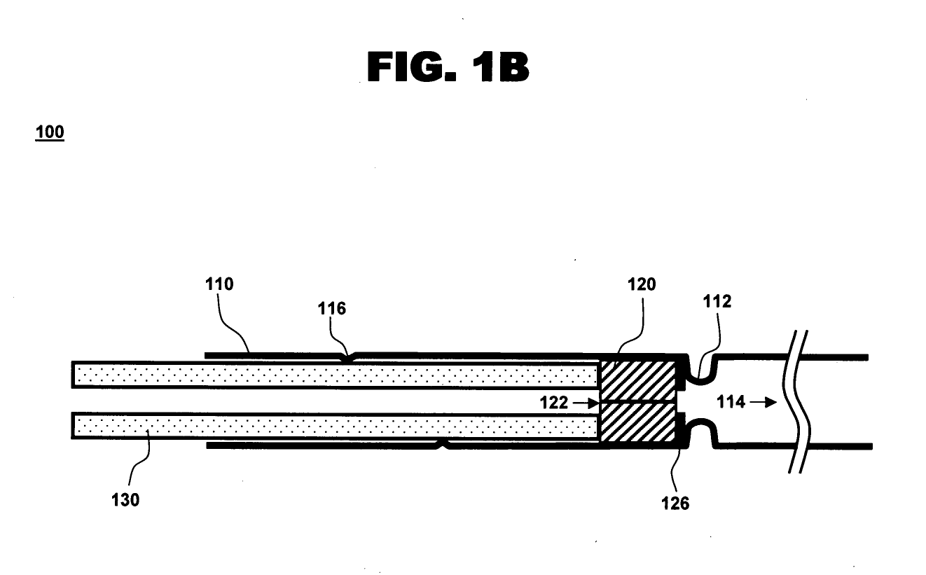

One aspect of the present invention is a low-profile catheter valve. One embodiment of the valve, in accordance with the present invention, is illustrated in FIGS. 1A and 1B at 100. Valve 100 comprises catheter 110, elastomeric plug 120, and hollow stem 130. Catheter 110 includes stop 112, which extends into central lumen 114 of catheter 110, and indentations 116. Plug 120 has a bore running through the plug to form channel 122, which is in fluid communication with central lumen 114. A perforated plate, washer 126, is also included in valve 100.

In the present embodiment, catheter 110 is a hypotube made of a biocompatible material such as stainless steel or nitinol. Catheter 110 may be a hollow guidewire and may include an inflatable balloon (not shown) operably attached to a distal portion of the catheter. Where catheter 110 is to be used as a guidewire during a procedure such as a conventional percutaneous transluminal coronary angioplasty involving femoral artery access, catheter 110 may be about 120 centimeters to about 300 centimeters long, with a length of about 180 centimeters often being used. The outer diameter of the catheter may range from about 0.010 inches to 0.038 inches, and preferably is 0.014 inches or smaller when the catheter is to be used as a guidewire. Thus, the outer diameter of catheter 110 provides a close sliding fit for a treatment instrument such as an atherectomy catheter, an angioplasty catheter, or a stent delivery catheter, which is inserted over valve 100 and advanced through vasculature.

Catheter 110 has stop 112 adjacent to the catheter's proximal end, i.e., the end that remains outside of the patient during a treatment procedure. The stop in the present embodiment is a single annular indentation swaged into catheter 110 and extending into central lumen 114. A series of crimped indentations spaced around the circumference of the catheter may also be used.

Elastomeric plug 120 is received within central lumen 114 of catheter 110 and is seated against stop 112. Thus, plug 120 is positioned within the lumen between stop 112 and the proximal end of catheter 110. Washer 126 is positioned between stop 112 and plug 120 to increase the surface area of the stop and improve the seating of plug 120 against stop 112. Plug 120 includes channel 122, which allows a fluid to pass through plug 120 into a distal portion of central lumen 114 when the plug is in an unstressed state as shown in FIG. 1A.

A distal portion of hollow stem 130 is slidably received within central lumen 114 of catheter 110. That is, stem 130 is passed into the proximal end of catheter 110 and seated against plug 120. FIG. 1A shows valve 100 in an open configuration wherein stem 130 applies little or no axial force against plug 120 such that channel 122 remains in its normally open form. To close valve 100, as shown in FIG. 1B, stem 130 is advanced into central lumen 114 until stem 130 applies an axial force against plug 120, axially compressing plug 120 against stop 112 and closing channel 122. Plug 120 is fabricated using an appropriate biocompatible material such as a silicone elastomer. The material is chosen to be both compressible when an axial force is applied via stem 130 and resilient to allow plug 120 to resume its unstressed configuration when the force is withdrawn. If a material is chosen that has a tendency to adhere to itself such that channel 122 remains closed after the force is withdrawn, a coating may be applied to the wall of channel 122 to prevent adherence.

Stem 130 is maintained in position within catheter 110 by an interference fit between stem 130 and central lumen 114. In the present embodiment, stem 130 is a nitinol or stainless steel hypotube having an outer diameter smaller than the diameter of central lumen 114. Interference between stem 130 and catheter 110 is provided by indentations 116 formed in the external wall of catheter 110 and extending into central lumen 114. These indentations contact the exterior of stem 130, increasing the frictional force that must be overcome to move stem 130 within the lumen.

The frictional force between stem 130 and catheter 110 must be sufficient to ensure that valve 100 is held in a closed configuration. Once stem 130 has been advanced into central lumen 114 to apply axial force against plug 120 and close channel 122, stem 130 is held in the closed position to maintain the axial force, thereby maintaining the plug in a compressed state and ensuring that valve 100 remains closed. That is, the frictional force between stem 130 and catheter 110 must be greater than the rebound force of elastomeric plug 120 plus any fluid pressure maintained within catheter 110 by plug 120. The axial force on plug 120 is removed by withdrawing stem 130, allowing plug 120 to resume its unstressed configuration with channel 122 open.

Where catheter 110 is to be used as a guidewire having an inflatable balloon at its distal end, channel 122 allows inflation or deflation of the balloon through central lumen 114. An axial force applied to stem 130 to compress plug 120 closes channel 122 to maintain inflation.

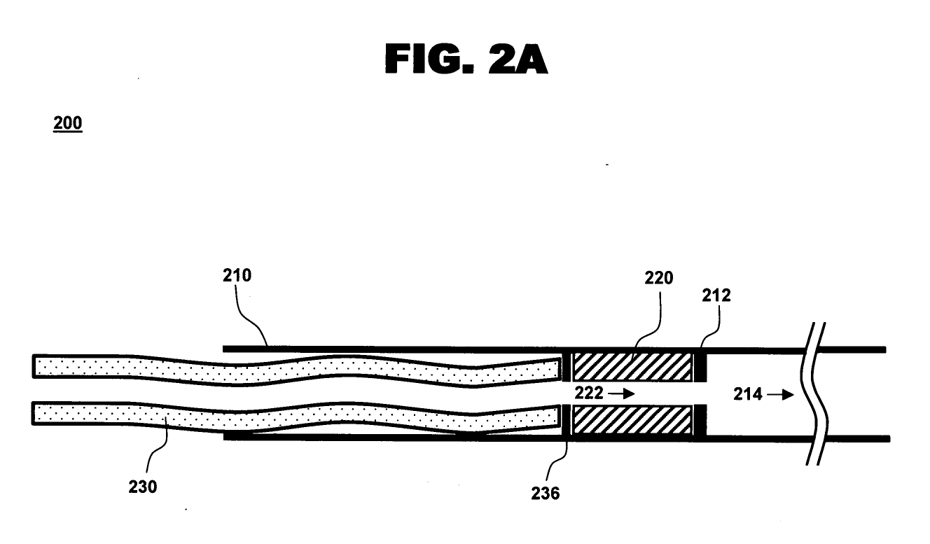

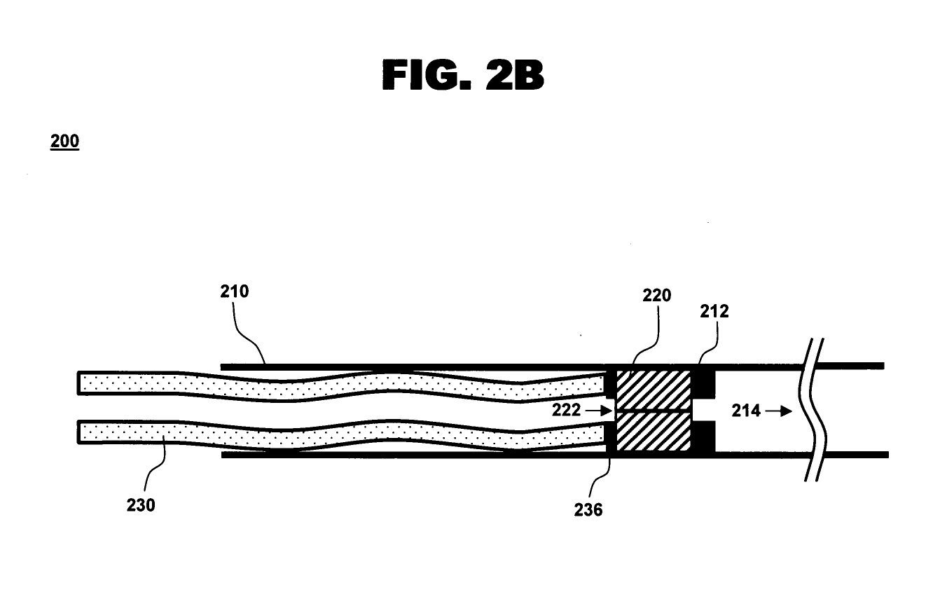

Another embodiment of the low-profile catheter valve, in accordance with the present invention, is illustrated in FIGS. 2A and 2B at 200. Valve 200 comprises catheter 210, elastomeric plug 220, and hollow stem 230. Catheter 210 includes stop 212, which extends into central lumen 214 of catheter 210. Plug 220 has a bore running through the plug to form channel 222, which is in fluid communication with central lumen 214. A perforated plate, washer 236, is also included in valve 200.

In the present embodiment, catheter 210 comprises a stainless steel or nitinol hypotube. A cylindrical structure is bonded by a method such as welding or adhesive bonding to the interior wall of catheter 210, extending into central lumen 214 to form stop 212.

Plug 220 comprises an appropriate biocompatible elastomer that is compressed when an axial force is applied to the plug via stem 230. Plug 220 is shown in an unstressed state in FIG. 2A with channel 222 open, and compressed in FIG. 2B with channel 222 closed. Washer 236 is positioned between plug 220 and stem 230 to increase the area of plug 220 to which the axial force is applied.

Stem 230 is maintained in position within catheter 210 by interference between the stem and the catheter. In the present embodiment, the interference between stem 230 and catheter 210 is provided by waves formed in stem 230. The waves contact the walls of central lumen 214, holding the stem in a desired position.

It will be apparent to one skilled in the art that interference between a stem and a catheter may be provided in a wide variety of ways, including varying the shape or size of either or both of the catheter and the stem, or by forming a variety of patterns of indentations in the external wall of the catheter. Indentations or other structures in the catheter and the stem may also be designed to interlock to provide the desired interference.

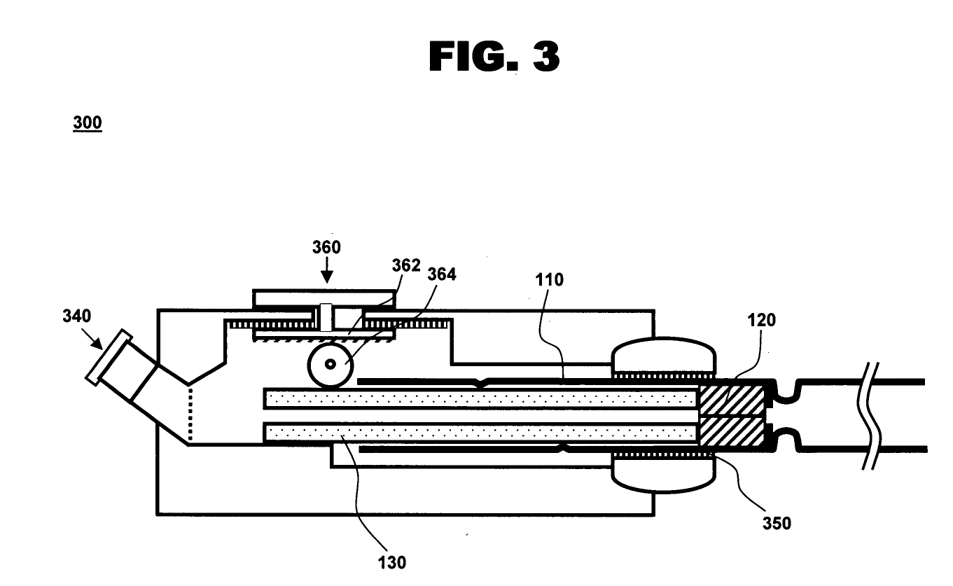

An adaptor may be used to apply and withdraw the axial force. FIG. 3 illustrates at 300 one embodiment of an adaptor in accordance with the present invention. The valve of FIGS. 1A and 1B is shown in place in the adaptor, with like elements sharing like numbers in FIGS. 1A and 1B and FIG. 3.

Adaptor 300 is shown removably mounted about a proximal portion of catheter 110. Adaptor 300 is movable between a first position in which hollow stem 130 applies an axial force to plug 120 and a second position in which the force is withdrawn. The adaptor is in fluid communication with a fluid delivery device through opening 340. Seal 350 engages the circumference of catheter 110 to establish a fluid-tight chamber surrounding the proximal end of catheter 110. Additional seals and gaskets may be used to ensure that the adaptor itself is fluid tight, especially around switch 360.

FIG. 3 shows adaptor 300 in the first position, with an axial force applied to plug 120 via stem 130. The axial force is supplied by means of switch 360, which includes rack 362 and internal wheel 364. Wheel 364 is in contact with a portion of stem 130 that extends beyond the proximal end of catheter 110. The wheel is fabricated using a material having a high coefficient of friction to engage the outer surface of stem 130 and communicate the axial force from switch 360 to stem 130.

Moving switch 360, and thereby rack 362, in the direction of the proximal end of catheter 110 rotates wheel 364 counterclockwise, providing a forward motion to stem 130 that compresses plug 120 and closes channel 122. The amount of axial force applied to plug 120 is predetermined by the axial freedom of movement of switch 360. Stem 130 is maintained in position by interference between the stem and the catheter. Thus, once stem 130 has been advanced to compress plug 120 and close channel 122, stem 130 is retained in the closed position, thereby maintaining plug 120 in a compressed state and ensuring that channel 122 remains closed. Adaptor 300 may then be removed to allow a treatment instrument to be advanced over catheter 110.

To reopen channel 122, adaptor 300 is again removably mounted about a proximal portion of catheter 110, and the direction of switch 360 is reversed, thereby providing a reverse motion to stem 130 that withdraws the axial compression force and allows plug 120 to resume its unstressed state with channel 122 open.

FIG. 3 shows just one example of an adaptor that may be used to operate valve 100. One skilled in the art will recognize that a wide variety of adaptors are appropriate for operating valve 100, valve 200, and other embodiments of a low-profile valve in accordance with the present of the invention.

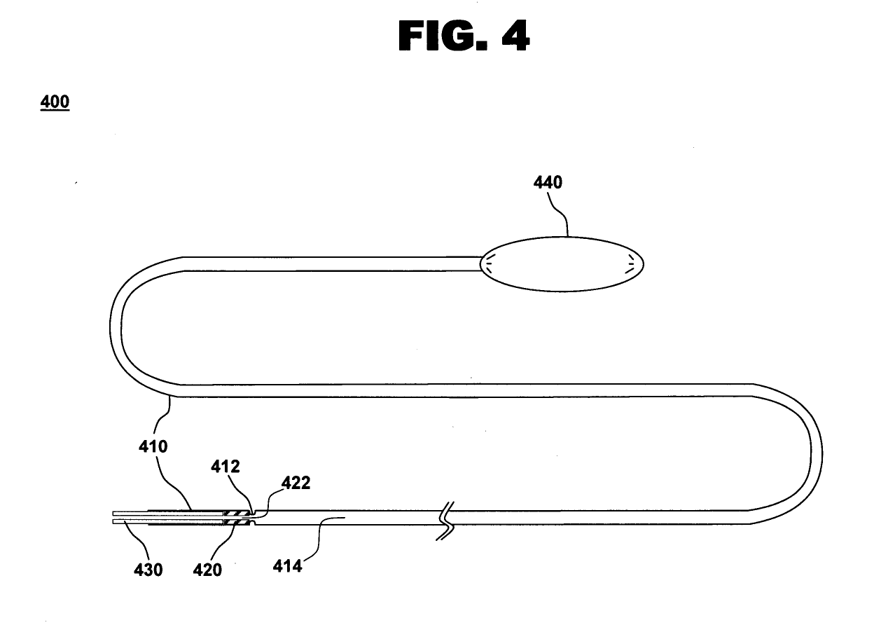

Another aspect of the present invention is a system for treating a vascular condition. One embodiment of the system, in accordance with the present invention, is illustrated in FIG. 4 at 400. System 400 comprises catheter 410, elastomeric plug 420, hollow stem 430, and inflatable balloon 440. Catheter 410 includes stop 412, which extends into central lumen 414 of catheter 410. Plug 420 has a bore running through the plug to form channel 422, which is in fluid communication with central lumen 414.

In the present embodiment, catheter 410 is a hollow guidewire comprising a hypotube made of a biocompatible material such as stainless steel or nitinol. Where catheter 410 is to be used as a guidewire for other catheters in a conventional percutaneous transluminal coronary angioplasty procedure involving femoral artery access, catheter 410 may be about 120 centimeters to about 300 centimeters long, with a length of about 180 centimeters often being used. The outer diameter of the catheter may range from about 0.010 inches to 0.038 inches, and preferably is 0.014 inches in outer diameter or smaller when used as a guidewire for other catheters. Thus, the outer diameter of catheter 410 provides a close sliding fit for a treatment instrument such as an atherectomy catheter, an angioplasty catheter, or a stent delivery catheter, which is inserted over system 400 and advanced through vasculature.

Catheter 410 has stop 412 extending into central lumen 414 adjacent to the catheter's proximal end, i.e., the end that remains outside of the patient during a treatment procedure. The stop in the present embodiment is an annular indentation swaged into catheter 410 and extending into central lumen 414. In an alternative embodiment, stop 412 may comprise multiple individual indentations or one or more structures bonded to the interior wall of the catheter.

Elastomeric plug 420 is received within central lumen 414 of catheter 410 and is seated against stop 412. Thus, plug 420 is positioned within the lumen between stop 412 and the proximal end of catheter 410. Hollow stem 430 is slidably received within central lumen 414 of catheter 410 and positioned against plug 420.

In the present embodiment, stem 430 is a nitinol or stainless steel hypotube having an outer diameter smaller than the inner diameter of catheter 410. When stem 430 is advanced within central lumen 414, the stem applies an axial force to compress plug 420 against stop 412, thus closing channel 422. Perforated plates (not shown) may be positioned between stop 412 and plug 420 and between plug 420 and stem 430, the first plate increasing the surface area of the stop and improving the seating of plug 420 against stop 412 and the second plate increasing the area of plug 420 to which the axial force is applied.

Plug 420 is fabricated using an appropriate biocompatible material such as a silicone elastomer. The material is chosen to be both compressible when an axial force is applied via stem 430 and resilient to allow plug 420 to resume its unstressed configuration when the force is withdrawn. If a material is chosen that has a tendency to adhere to itself such that channel 422 remains closed after the force is withdrawn, a coating may be applied to the wall of channel 422 to prevent adherence.

Stem 430 is maintained in position within catheter 410 by interference between the stem and the catheter. Interference between stem 430 and catheter 410 is provided by indentations formed in the external wall of catheter 410 and extending into the central lumen. These indentations contact the exterior of stem 430, increasing the frictional force that must be overcome to move stem 430 within the lumen. It will be apparent to one skilled in the art that interference between a stem and a catheter may be provided in a wide variety of ways. For example, a single annular indentation may be formed in the external wall of the catheter, the indentation extending into the central lumen of the catheter. Alternatively, a variety of catheter and stem shapes and sizes may be used to achieve interference between the catheter and stem.

The frictional force between stem 430 and catheter 410 must be sufficient to ensure that the valve is held in a closed configuration. Once stem 430 has been advanced into central lumen 414 to apply axial force against plug 420 and close channel 422, stem 430 is held in the closed position to maintain the axial force, thereby maintaining the plug in a compressed state and ensuring that the valve remains closed. That is, the frictional force between stem 430 and catheter 410 must be greater than the rebound force of elastomeric plug 420 plus any fluid pressure maintained within catheter 410 by plug 420. The axial force on plug 420 is removed by withdrawing stem 430, allowing plug 420 to resume its unstressed configuration with channel 422 open.

Inflatable balloon 440 is operably attached to a distal portion of catheter 410. Inflatable balloon 440 may be made of a suitable material such as thermoplastic polyurethane (TPU) resins, styrene-ethylene-butadiene-styrene (SEBS), PEBAX, or the like. Channel 422 allows inflation of the balloon through central lumen 414. Inflation is maintained when an axial force is applied to stem 430 to compress plug 420 and close channel 422. Withdrawing the axial force allows plug 420 to resume its unstressed configuration with channel 422 reopened to permit deflation of balloon 440.

The system as depicted may be operated using an adaptor such as that shown in FIG. 3. The adaptor may be removably mounted about a proximal portion of catheter 410. The adaptor has a seal to engage the circumference of catheter 410. Engaging the seal establishes a fluid-tight chamber surrounding the proximal end of catheter 410. The adaptor is movable between a first position in which hollow stem 430 applies an axial force to plug 420 and a second position in which the force is withdrawn. The adaptor is in fluid communication with a fluid delivery device and has a seal to engage the circumference of catheter 410. When engaged, the seal establishes a fluid-tight chamber surrounding the proximal end of catheter 410. Additional seals and gaskets may be used to ensure that the adaptor itself is fluid tight. Mechanical stops or other means may be provided to ensure the adaptor applies a predetermined axial force.

Although described above in the context of an occlusion guidewire, system 400 may be readily adapted to a wide variety of balloon catheters, including those having additional functionalities, structures, or intended uses.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

1. A low-profile catheter valve, comprising:
a catheter having at least one stop extending into a central lumen of the catheter adjacent to a proximal end of the catheter;
an elastomeric plug having a channel in fluid communication with the central lumen of the catheter, the plug received within the central lumen and seated against the stop; and
a hollow stem, at least a portion of the stem slidably received within the central lumen of the catheter, wherein an axial force applied to the elastomeric plug via the stem compresses the plug and closes the channel, and wherein the stem is maintained in a position within the catheter by interference between the stem and the catheter.

2. The valve of claim 1 wherein the catheter is a hollow guidewire.

3. The valve of claim 1 wherein the catheter is sized such that it provides a close sliding fit for a treatment instrument that is advanceable over the catheter and through a vasculature.

4. The valve of claim 1 wherein the catheter is one of a stainless steel or a nitinol hypotube.

5. The valve of claim 1 wherein the catheter stop comprises at least one indentation formed in an external wall of the catheter, the indentation extending into the central lumen.

6. The valve of claim 1 wherein the catheter stop comprises at least one structure bonded to an internal wall of the catheter, the structure extending into the central lumen.

7. The valve of claim 1 wherein the interference between the stem and the catheter is provided by waves formed in the hollow stem.

8. The valve of claim 1 wherein the interference between the stem and the catheter is provided by at least one indentation formed in an external wall of the catheter, the indentation extending into the central lumen.

9. The valve of claim 1 wherein a portion of the hollow stem extends beyond a proximal end of the catheter.

10. The valve of claim 1 further comprising:
a perforated plate positioned between the catheter stop and the elastomeric plug.

11. The valve of claim 1 further comprising:
a perforated plate positioned between the elastomeric plug and the hollow stem.

12. The valve of claim 1 further comprising:
a coating disposed on a wall of the channel.

13. The valve of claim 1 further comprising:
an adaptor removably mounted about a proximal portion of the catheter, the adaptor being movable between a first position in which the hollow stem applies an axial force to the elastomeric plug and a second position in which the force is withdrawn, the adaptor being in fluid communication with a fluid delivery device, the adaptor having a seal to engage the circumference of the catheter, wherein engaging the seal establishes a fluid-tight chamber surrounding a proximal end of the catheter.

14. The valve of claim 13 wherein the adaptor applies a predetermined axial force.

15. The valve of claim 1 wherein an inflatable balloon is operably attached to a distal portion of the catheter, the elastomeric plug channel allowing inflation or deflation of the balloon through the central lumen of the catheter, the axial force applied to the elastomeric plug via the stem closing the channel to maintain inflation of the balloon.

16. A system for treating a condition in a patient's vasculature, the system comprising:
a catheter having at least one stop extending into a central lumen of the catheter adjacent to a proximal end of the catheter;
an elastomeric plug having a channel in fluid communication with the central lumen of the catheter, the plug received within the central lumen and seated against the stop;
a hollow stem, at least a portion of the stem slidably received within the central lumen of the catheter; and
an inflatable balloon operably attached to a distal portion of the catheter, wherein the channel of the elastomeric plug allows inflation or deflation of the balloon through the central lumen of the catheter, and wherein an axial force applied to the elastomeric plug via the stem compresses the plug and closes the channel to maintain inflation, the stem being maintained in a position within the catheter by interference between the stem and the catheter.

17. The system of claim 16 wherein the catheter is a hollow guidewire.

18. The system of claim 16 wherein the catheter is sized such that it provides a close sliding fit for a treatment instrument that is advanceable over the catheter and through the vasculature.

19. The system of claim 16 wherein the catheter is one of a stainless steel or a nitinol hypotube.

20. The system of claim 16 wherein the catheter stop comprises at least one indentation formed in an external wall of the catheter, the indentation extending into the central lumen.

21. The system of claim 16 wherein the catheter stop comprises at least one structure bonded to an internal wall of the catheter, the structure extending into the central lumen.

22. The system of claim 16 wherein the interference between the stem and the catheter is provided by waves formed in the hollow stem.

23. The system of claim 16 wherein the interference between the stem and the catheter is provided by at least one indentation formed in an external wall of the catheter, the indentation extending into the central lumen.

24. The system of claim 16 wherein a portion of the hollow stem extends beyond a proximal end of the catheter.

25. The system of claim 16 further comprising:
a perforated plate positioned between the catheter stop and the elastomeric plug.

26. The system of claim 16 further comprising:
a perforated plate positioned between the elastomeric plug and the hollow stem.

27. The system of claim 16 further comprising:
a coating disposed on a wall of the channel.

28. The system of claim 16 further comprising:
an adaptor removably mounted about a proximal portion of the catheter, the adaptor being movable between a first position in which the hollow stem applies an axial force to the elastomeric plug and a second position in which the force is withdrawn, the adaptor being in fluid communication with a fluid delivery device, the adaptor having a seal to engage the circumference of the catheter, wherein engaging the seal establishes a fluid-tight chamber surrounding a proximal end of the catheter.

29. A low-profile catheter valve, comprising:
a catheter;
compressible means for providing a closed fluid path when an axial force is applied to the compressible means and an open fluid path when the axial force is removed, the compressible means positioned within a central lumen of the catheter; and
means for applying the axial force to the compressible means, the application means slidably received within the central lumen of the catheter.

30. The valve of claim 29 further comprising:
means for placing the catheter in fluid communication with a fluid delivery device.
